(19) United States
(12) Patent Application Publication
(10) Pub. No.: US 2003/0232887 A1
(43) Pub. Date: Dec. 18, 2003

(54) Preparation and use of a stable formulation of allosteric effector compounds

(76) Inventors: Douglas Giles Johnson, Arvada, CO (US); Mark Doty, Grayslake, IL (US); James E. Kipp, Wauconda, IL (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/120,848

(22) Filed: Apr. 10, 2002

(51) Int. Cl. A61K 31/195; A61K 9/14

(52) U.S. Cl. 514/567

(57) ABSTRACT

A pharmaceutical composition of 2-[4-[2-[(3,5-dimethylphenyl)amino]-2-oxoethyl]phenoxy]-2-methyl-propionic acid or its physiologically acceptable salts suitable for parenteral administration includes an aqueous formulation of 2-[4-[2-[(3,5-dimethylphenyl)amino]-2-oxoethyl]phenoxy]-2-methyl-propionic acid or its physiologically acceptable salt and a buffer to maintain the pH from about 6 to about 11. The composition in accordance with the invention reduces the amount of particulate matter that forms in solution after heat sterilization. The invention also includes a process for making a pharmaceutical composition of 2-[4-[2-[(3,5-dimethylphenyl)amino]-2-oxoethyl]phenoxy]-2-methyl-propionic acid or its physiologically acceptable salt that has a shelf life in excess of thirty days and is useful in parenteral administration.

### FIELD OF THE INVENTION

The present invention is directed to a pharmaceutical preparation of allosteric effector compounds or their physiologically acceptable salts. More particularly, the invention includes a stable composition of 2-[4-[2-[(3,5-dimethylphenyl)amino]-2-oxoethyl]phenoxy]-2-methyl-propionic acid or its physiologically acceptably salt.

### BACKGROUND OF THE INVENTION

It has been found that a family of compounds including the specific compound 2-[4-[2-[(3,5-dimethylphenyl)amino]-2-oxoethyl]phenoxy]-2-methyl-propionic acid are allosteric modifiers of hemoglobin. This property is useful in vitro and in vivo in methods using the compounds for allosterically modifying hemoglobin, for storing blood, for treating blood such that the hemoglobin in said blood is allosterically modified towards a low oxygen affinity state, and for restoring the oxygen affinity of red blood cells.

The ability to allosterically modify hemoglobin also allows the compounds to be useful in treating a variety of disorders and conditions mediated through allosterically modifying hemoglobin to shift oxygen equilibrium in favor of free oxygen. Such disorders may include, but are not limited to, whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, oxygen deprivation of a fetus. The compound is useful in a method comprising the step of administering to a patient suffering from or undergoing the claimed condition a sufficient quantity of an allosteric effector compound. In the case of carcinogenic tumors, the compounds are useful alone, and as radiosensitizers in conduction with ionizing radiation. The allosteric modification properties also allow it to be useful in certain imaging methods, especially blood oxygen level dependent MRI, and also in diagnostic methods, including determination of tumor oxygenation, and determination of an optimal time for commencing radiation treatment based on tumor oxygenation. The preparation and uses for 2-[4-[2-[(3,5-dimethylphenyl)amino]-2-oxoethyl]phenoxy]-2-methyl-propionic acid and its physiologically acceptable salts has been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and pending U.S. patent application Ser. No. 10/082,130, filed Feb. 25, 2002. These patents also describe the preparation and use of structurally similar compounds. Other patents describing closely related compounds include 5,248,785; 5,731,454. These patents, applications, and all other patents, applications, and publications referred to herein, including the USP 25 <788>, are specifically incorporated by reference herein. As used herein, [2-[(3,5-dimethylphenyl)amino]-2-oxoethyl]phenoxy]-2-methyl-propionic acid and its physiologically acceptably salts will be collectively referred to as an “allosteric modifying compound.” The most convenient form of the allosteric modifying compound for intravenous injection, continuous infusion, or other parenteral routes is one that is sterile and ready to administer without any mixing, admixing, filtering, or other steps.

### SUMMARY OF THE INVENTION

The present invention provides stabilized pharmaceutical compositions comprising an allosteric modifier compound and a stabilizing compound.

The allosteric effector compounds useful in the invention are, a compound having the formula:

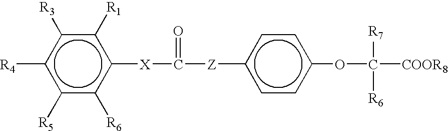

where R₁₋₅ may be hydrogen, halogen, or a substituted or unsubstituted C₁₋₃ alkyl group and may be the same or different,

R₆₋₇ may each be hydrogen or methyl and may be the same or different, and

R₈ may be hydrogen, a substituted or unsubstituted C₁₋₃ alkyl group, or a salt cation, and

X and Z are CH₂, NH, or O;

a compound having the formula:

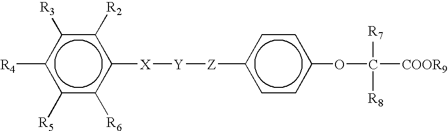

where X and Z may each be CH₂, CO, NH or O, and Y may be CO or NH, which the caveat that X, Y, and Z must all be different from each other, and

R₂₋₆ can be the hydrogen, halogen, substituted or unsubstituted C₁₋₃ alkyl groups, and may be the same or different,

R₇₋₈ can be hydrogens, methyls, ethyls, or alkyl groups in a ring connecting the two, and

R₉ can be a hydrogen, lower alkyl, or salt cation;

a compound having the formula:

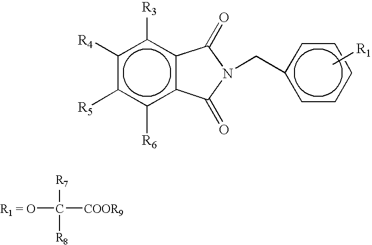

where R₃₋₆ can be the hydrogen, halogen, substituted or unsubstituted C₁₋₃ alkyl group, or a C₁₋₃ ether or ester, and these moieties may be the same or different, or alkyl moieties of an aromatic or aliphatic ring incorporating two of the R₃₋₆,

R₁ can be connected to any position on the phenyl ring, and

sites R₇₋₈ can be hydrogen, halogen, methyl, ethyl, and these moieties may be the same or different, or alkyl groups in a ring connecting the two, and

R₉ can be a hydrogen, halogen, C₁₋₃ lower alkyl, or salt cation;

a compound having the formula:

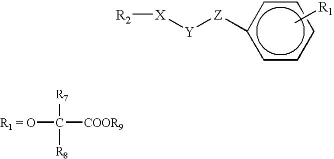

where R₁ can be connected to any position on the phenyl ring, and

sites R₇₋₈ can be hydrogen, halogen, methyl, ethyl, and these moieties may be the same or different, or alkyl groups in a ring connecting the two, and

R₂ is defined as a substituted or unsubstituted aromatic compound, a substituted or unsubstituted alkyl ring compound, or a substituted or unsubstituted phthalimide compound,

X is a carboxyl,

Y is a nitrogen,

and R₂ completes the phthalimide compound by being bonded to both X and Y; and

where X, Y, and Z, may either be CH₂, NH, O, or N, with the caveat that each are different from the other;

a compound having the formula:

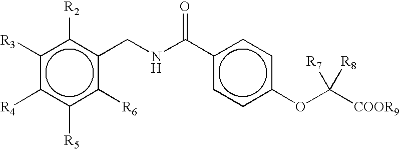

where R₂, R₃, R₄, R₅, and R₆ may be hydrogen, halogen, or alkyl groups and may be the same or different,

R₇ and R₈ may be hydrogen or methyl groups and may be the same or different, and

where the R₉ moiety is hydrogen or a salt cation;

a compound having the formula:

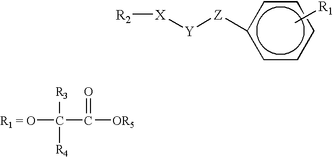

where R₂ is a substituted or unsubstituted aromatic compound, or a substituted or unsubstituted alkyl ring compound, or a substituted or unsubstituted phthalimide compound that incorporates X and Y,

X is a carbonyl,

Y is a nitrogen, and

R₂ completes the phthalimide compound by being bonded to both X and Y, and where X, Y, and Z are CH₂, NH, S, SO₂, CO, O or N with the caveat that X, Y, and Z are each different from one another, and

where R₁ can be connected to any position on the phenyl ring, and

R₃ and R₄ are hydrogen, halogen, methyl, ethyl, propyl, isopropyl, neopentyl, butyl, or substituted or unsubstituted aryl groups and these moieties may be the same or different, or alkyl moieties as part of an aliphatic ring connecting R₃ and R₄, and

R₅ is a hydrogen, halogen, C₁₋₃ lower alkyl, or a salt cation;

a compound having the formula:

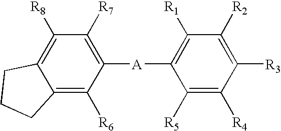

where A is a chemical bridge which includes two to four chemical moieties bonded together,

the chemical moieties in A are selected from the group consisting of CO, O, S, SO₂, NH, NR₉ where R₉ is a C₁₋₆ alkyl group, CH₂, CH, and C, with the proviso that, except in the case where A contains two identical CH and C moieties positioned adjacent one another to form an alkene or alkyne, the chemical moieties in A are each different from one another, and

at least one of R₁₋₅ is substituted with a compound having the chemical formula:

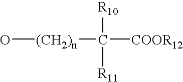

where n is zero to five,

where R₁₀ and R₁₁, are selected from the group consisting of hydrogen, halogen, C₁₋₁₂ alkyl groups, carboxylic acids and esters, aromatic or heteroatomic groups, and these moieties may be the same or different, or alkyl moieties of part of an aliphatic ring connecting R₁₀ and R₁₁, and where R₁₂ is a hydrogen, halogen, salt cation, metal, or C₁₋₆ alkyl group, and

wherein a remainder of the R₁₋₅ moieties and the R₆₋₈ moieties are selected from the group consisting of hydrogen, halogen, C₁₋₆ alkyl groups, C₁₋₆ ether or esters, aromatics and heteroaromatics, and alkyl moieties of an aliphatic ring connecting two sites on a phenyl group;

a compound having the formula:

R₁—A—R₂

where R₁ and R₂ each are a substituted or unsubstituted aromatic or heteroaromatic compounds, or a substituted or unsubstituted alkyl or heteroalkyl ring compound, or a substituted or unsubstituted phthalimide compound, and

where R₁ and R₂ may be the same or different,

where A is a chemical bridge which includes three chemical moieties bonded together between R₁ and R₂,

wherein the chemical moieties in A are selected from the group consisting of CO, O, S, SO₂, NH, NR₃ where R₃ is C₁₋₆ alkyl group, NR₄ where R₄ includes two carbonyls as part of a phthalimide compound formed with R₁ or R₂, CH₂, CH, and C, and

where at least one of R₁ and R₂ is substituted with a compounds having the chemical formula:

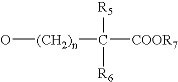

where n is zero to five, where R₅ and R₆ are selected from the group consisting of hydrogen, halogen, substituted or unsubstituted C₁₋₁₂ alkyl groups, carboxylic acid and ester groups, substituted or unsubstituted aromatic or heteroaromatic groups, and these moieties may be the same or different, or alkyl moieties of part of an aliphatic ring connecting R₅ and R₆, and

where R₇ is a hydrogen, halogen, salt cation, metal, or substituted or unsubstituted C₁₋₆ alkyl group;

a compound having the formula:

R₁—A—R₂

where R₁ and R₂ each are a substituted or unsubstituted aromatic or heteroaromatic compound, or substituted or unsubstituted alkyl or heteroalkyl ring compound, or a substituted or unsubstituted phthalimide compound, and

where R₁ and R₂ may be the same or different,

where A is a chemical bridge which includes two to four chemical moieties bonded together between R₁ and R₂,

wherein said chemical moieties in A are selected from the group consisting of CO, O, S, SO₂, NH, NR₃ where R₃ is a C₁₋₆ alkyl group, NR₄ where R₄ includes two carbonyls as part of a phthalimide compound formed with R₁ or R₂, CH₂, CH, and C, with the caveat that, except in the case where A contains two identical CH and C moieties positioned adjacent one another to form an alkene or alkyne, the chemical moieties in A are each different from one another, and

wherein at least one of R₁ or R₂ is substituted with a compound having the chemical formula:

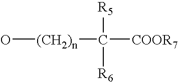

where n is zero to five,

where R₅ and R₆ are selected from the group consisting of hydrogen, halogen, substituted or unsubstituted C₁₋₁₂ alkyl groups, carboxylic acid and ester, substituted or unsubstituted aromatic or heteroaromatic groups, and these moieties may be the same or different, or alkyl moieties of part of an aliphatic ring connecting R₅ and R₆, and

where R₇ is a hydrogen, halogen, salt cation, metal, or substituted or unsubstituted C₁₋₆ alkyl group; and/or

a compound having the formula:

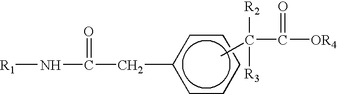

where R₁ is selected from the group consisting of optionally substituted phenyl, adamantyl, napthyl, and indanyl, R₂₋₃ are alkyl moieties of a C₃₋₆ alkyl ring connecting R₂ and R₃, and R₄ is a hydrogen, a monovalent salt cation, or a C₁₋₃ lower alkyl.

In some embodiments, the allosteric effector compound is 2-[4-(((3,5-dimethylanilino)carbonyl)methyl)phenoxy]-2-methylpropionic acid, or a physiologically acceptable salt thereof.

In preferred embodiments, the allosteric effector compound is 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid.

In some embodiments, the composition has, as measured by light obscuration per USP 25 <788>, not more than 3 particles per milliliter of particles ≧95 μm and not more than 25 particles per milliliter of particles ≧10 μm, while in other embodiments, the composition has, as measured by light obscuration per USP 25 <788>, not more than 600 particles per container of particles ≧25 μm, or not more than 6000 particles per container of particles ≧10 μm.

Preferably, the composition includes an amount of allosteric effector compound from about 15 millimoles/L to about 120 millimoles/L.

The stabilizing agent is selected from the group consisting of orthophosphoric acid, physiologically acceptable salts of orthophosphoric acid, citric acid, physiologically acceptable salts of citric acid, tromethamine, meglumine, amino acids, di-peptides, tri-peptides, glycine, lysine, arginine, glycyl-glycine, and combinations thereof.

The allosteric effector compound is present as a physiologically acceptable salt selected from the group consisting of sodium, potassium, calcium, magnesium, zinc, and combinations thereof, in some embodiments. In further embodiments, the physiologically acceptable salt is a salt of a compound containing an amine group. In other embodiments, the compound containing a free amino group is selected from the group consisting of lysine, hydroxy-lysine, histidine, arginine, ornithine, protonated tromethamine, meglumine, and combinations thereof.

The composition contains an amount of 2-[4-[2-[(3,5-dimethylphenyl)amino]-2-oxoethyl]phenoxy]-2-methyl-propionic acid effective for the treatment of conditions mediated through allosterically modifying hemoglobin to shift oxygen equilibrium in favor of free oxygen. In some embodiments, the physiologically acceptable salt of the allosteric effector compound comprises a counter ion, which acts as the stabilizing agent.

In some embodiments, the composition is sterile. In other embodiments, the composition comprises a diluent such as water, a saline solution, a dextrose solution, lactated Ringer's solution, an aqueous solution of mannitol, or combinations thereof.

The present invention also provides a process of making a pharmaceutical composition of an allosteric effector compound, comprising the steps of combining allosteric effector compound or at least one physiologically acceptable salt thereof and a stabilizing agent. In some embodiments, the allosteric effector compound is provided in a diluent, and in further embodiments, the diluent has a pH above about 6.6.

In some embodiments, the stabilizing agent is added in amount sufficient to minimize the formation of particulates in the pharmaceutical composition, for example, maintaining the composition having not more than 3 particles per milliliter of particles ≧25 μm and not more than 25 particles per milliliter of particles ≧10 μm; or maintaining the composition having not more than 600 particles per container of particles ≧25 μm and not more than 6000 particles per container of particles ≧10 μm.

In some embodiments the stabilizing agent maintains the pH of the composition from about 6.5 to about 11. In other embodiments, the allosteric effector compound is added in an amount ranging from about 15 millimoles/L to about 120 millimoles/L.

In other embodiments, the counterions and stabilizing agents used are those described for the stabilized compositions of the invention.

In some embodiments, the method further provides for sterilizing the composition, for example, by heat sterilization, by sterile filling the composition into a sterile container.

### DETAILED DESCRIPTION OF THE INVENTION

The present invention includes pharmaceutically stabilized compositions of allosteric effector compounds. As used herein, “pharmaceutically stabilized allosteric effector compounds” refers to allosteric effector compounds maintained without the formation of substantial particulate matter. As used herein, lack of formation of substantial particulate matter, or minimization of formation of particulate matter refers to a level of particulate matter that makes the compound suitable for parenteral administration as defined in the United States Pharmacopeia monograph <788> (USP <788>). (United States Pharmacopeial Convention Committee of Revision, The United States Pharmacopeia, (25th edition)). This stabilization may be effected by the addition of one or more agents which, together with the allosteric effector compound, provide a pharmaceutical formulation which is capable of delivering the allosteric effector compound. In some embodiments the pharmaceutically stabilized composition includes a diluent in which the composition is prepared, or into which the composition is added.

Allosteric effector compounds which may be used in the formulations of the present invention fall into a number of different categories:

Group I: 2-[4-((aryl)acetamido)phenoxy]2-methyl propionic acid compounds having the general structural formula:

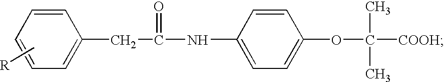

group II: 2-[4-(((aryl)oxy)carbonyl)amino) phenoxy]-2-methyl propionic acid compounds having the general structural formula

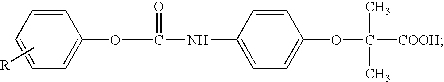

group III: 2-[4 ((((aryl)amino)carbonyl) methyl)phenoxy]-2-methyl propionic acid compounds having the general structural formulae

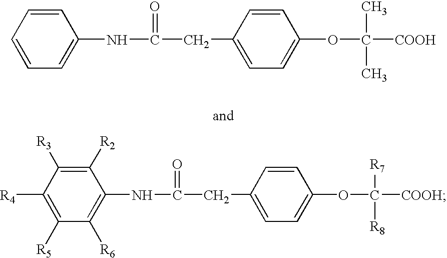

group IV: 2-[4-“-(((aryl)oxy)carbonyl)amino)phenoxy]-2-methyl propionic acid compounds having the general structural formula

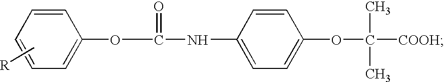

In one subset of compounds defined by the formula

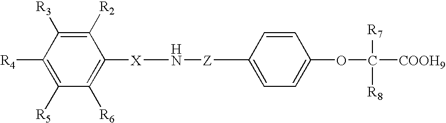

X and Z may each be CO or CH₂, with the caveat that when X is CO, Z is CH₂, and when X is CH₂, Z is CO. This subset of compounds may be conveniently divided into two additional groupings as follows:

Group V: 2-[4-(((aryloyl)amino) methyl)phenoxy]-2-methyl propionic acid compounds having the general structural formula

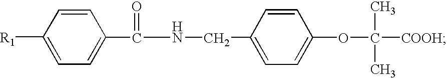

Group VI: 2-[4-((((aryl)methyl)amino) carbonyl)phenoxy]-2-methyl propionic acid compounds having the general structural formula; and

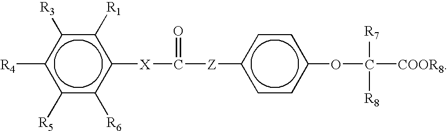

Group VII has the general structural formula:

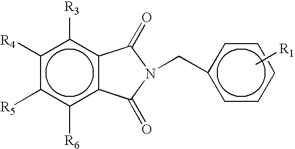

The image enhancing agents of the present invention are capable of allosterically effecting hemoglobin to cause a change in the oxy-/deoxy-hemoglobin ratio. Allosteric effector compounds useful in the present invention include compounds disclosed in U.S. Pat. No. 5,049,695, including

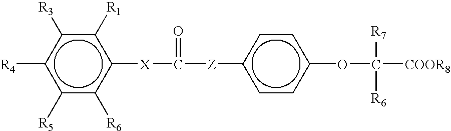

where R₁₋₅ may be hydrogen, halogen, or a substituted or unsubstituted C₁₋₃ alkyl group and may be the same or different, wherein R₆₋₇ may each be hydrogen or methyl and may be the same or different, and wherein R₈ may be hydrogen, a substituted or unsubstituted C₁₋₃ alkyl group, or a salt cation, and where X and Z are CH₂, NH, or O. Other allosteric effector compounds useful in the present invention disclosed in U.S. Pat. No. 5,122,539 include

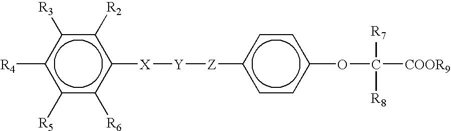

where X and Z may each be CH₂, CO, NH or O, and Y may be CO or NH, which the caveat that X, Y, and Z must all be different from each other. R₂₋₆ can be the hydrogen, halogen, substituted or unsubstituted C₁₋₃ alkyl groups, and may be the same or different, R₇₋₈ can be hydrogens, methyls, ethyls, or alkyl groups in a ring connecting the two, and R₉ can be a hydrogen, lower alkyl, or salt cation.

Also included as allosteric effector compounds useful in the present invention are compounds disclosed in U.S. Pat. No. 5,248,785 and U.S. Pat. No. 5,250,701, including

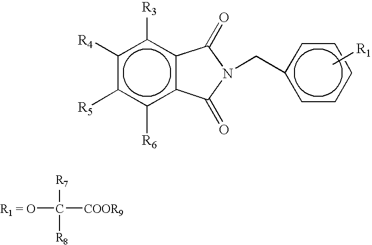

where R₃₋₆ can be the hydrogen, halogen, substituted or unsubstituted C₁₋₃ alkyl group, or a C₁₋₃ ether or ester, and these moieties may be the same or different, or alkyl moieties of an aromatic or aliphatic ring incorporating two of the R₃₋₆, and where R₁ can be connected to any position on the phenyl ring, and sites R₇₋₈ can be hydrogen, halogen, methyl, ethyl, and these moieties may be the same or different, or alkyl groups in a ring connecting the two, and R₉ can be a hydrogen, halogen, C₁₋₃ lower alkyl, or salt cation.

Also included as allosteric effector compounds useful in the present invention are compounds disclosed in U.S. Pat. No. 5,290,803 including

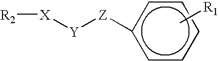

where R₁ is a tail structure as defined above in connection with U.S. Pat. No. 5,248,785, and R₂ is defined as a substituted or unsubstituted aromatic compound, a substituted or unsubstituted alkyl ring compound, or a substituted or unsubstituted phthalimide compound X is a carboxyl, Y is a nitrogen and R₂ completes the phthalimide compound by being bonded to both X and Y; and where X, Y, and Z, may either be CH₂, NH, O, or N, with the caveat that each are different from the other.

Also included as allosteric effector compounds useful in the present invention are compounds disclosed in U.S. Pat. No. 5,382,680 including

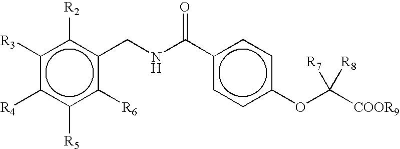

wherein the R₂, R₃, R₄, R₅, and R₆ moieties may be hydrogen, halogen, or alkyl groups and may be the same or different, wherein the R₇ and R₈ moieties may be hydrogen or methyl groups and may be the same or different, and wherein the R₉ moiety is hydrogen or a salt cation.

Also included as allosteric effector compounds useful in the present invention are compounds disclosed in U.S. Pat. No. 5,432,191 including

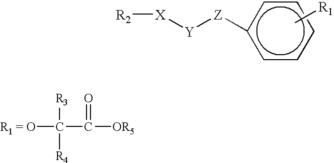

where R₂ is a substituted or unsubstituted aromatic compound, or a substituted or unsubstituted alkyl ring compound, or a substituted or unsubstituted phthalimide compound that incorporates X and Y where X is a carbonyl, Y is a nitrogen and R₂ completes the phthalimide compound by being bonded to both X and Y, and where X, Y, and Z are CH₂, NH, S, SO₂, CO, O or N with the caveat that the X, Y, and Z moieties are each different from one another, and where R₁ can be connected to any position on the phenyl ring, and R₃ and R₄ are hydrogen, halogen, methyl, ethyl, propyl, isopropyl, neopentyl, butyl, or substituted or unsubstituted aryl groups and these moieties may be the same or different, or alkyl moieties as part of an aliphatic ring connecting R₃ and R₄, and R₅ is a hydrogen, halogen, C₁₋₃ lower alkyl, or a salt cation.

Also included as allosteric effector compounds useful in the present invention are compounds disclosed in U.S. Pat. No. 5,591,892 including

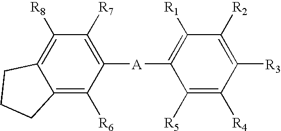

where A is a chemical bridge which includes two to four chemical moieties bonded together, wherein the chemical moieties in A are selected from the group consisting of CO, O, S, SO₂, NH, NR₉ where R₉ is a C₁₋₆ alkyl group, CH₂, CH, and C, with the proviso that, except in the case where A contains two identical CH and C moieties positioned adjacent one another to form an alkene or alkyne, the chemical moieties in A are each different from one another, and wherein at least one of R₁₋₅ is substituted with a compound having the chemical formula:

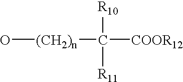

where n is zero to five, where R₁₀ and R₁₁ are selected from the group consisting of hydrogen, halogen, C₁₋₁₂ alkyl groups, carboxylic acids and esters, aromatic or heteroatomic groups, and these moieties may be the same or different, or alkyl moieties of part of an aliphatic ring connecting R₁₀ and R₁₁, and where R₁₂ is a hydrogen, halogen, salt cation, metal, or C₁₋₆ alkyl group, and wherein a remainder of the R₁₋₅ moieties and the R₆₋₈ moieties are selected from the group consisting of hydrogen, halogen, C₁₋₆ alkyl groups, C₁₋₆ ether or esters, aromatics and heteroaromatics, and alkyl moieties of an aliphatic ring connecting two sites on a phenyl group.

Also included as allosteric effector compounds useful in the present invention are compounds disclosed in U.S. Pat. No. 5,648,375 including a compound of the formula R₁—A—R₂ where R₁ and R₂ each are a substituted or unsubstituted aromatic or heteroaromatic compounds, or a substituted or unsubstituted alkyl or heteroalkyl ring compound, or a substituted or unsubstituted phthalimide compound, and where R₁ and R₂ may be the same or different, where A is a chemical bridge which includes 3 chemical moieties bonded together between R₁ and R₂, wherein the chemical moieties in A are selected from the group consisting of CO, O, S, SO₂, NH, NR₃ where R₃ is C₁₋₆ alkyl group, NR₄ where R₄ includes two carbonyls as part of a phthalimide compound formed with R₁ or R₂, CH₂, CH, and C, and where at least one of R₁ and R₂ is substituted with a compounds having the chemical formula:

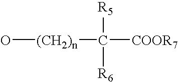

where n is zero to five, where R₅ and R₆ are selected from the group consisting of hydrogen, halogen, substituted or unsubstituted C₁₋₁₂ alkyl groups, carboxylic acid and ester groups, substituted or unsubstituted aromatic or heteroaromatic groups, and these moieties may be the same or different, or alkyl moieties of part of an aliphatic ring connecting R₅ and R₆, and where R₇ is a hydrogen, halogen, salt cation, metal, or substituted or unsubstituted C₁₋₆ alkyl group.

Also included as allosteric effector compounds useful in the present invention are compounds disclosed in U.S. Pat. No. 5,661,182, including an allosteric effector molecule which (i) binds to only one pair of symmetry related sites in the central water cavity of hemoglobin at the Lys 99 α, Arg 141 α, and Asn 108 β residues, each pair of symmetry related sites having residues on three separate sub-units of the hemoglobin, (ii) stabilizes the hemoglobin in a lower oxygen affinity state, and (iii) is active in the presence of normal concentrations of serum albumin in the blood, the allosteric effector molecule (a) maintains greater than sixty percent of its activity in terms of right shifting the oxygen dissociation curve of hemoglobin for a buffered red cell suspension at pH 7.4, in 140 mM NaCl and 50 mM bis-Tris buffer at 37° C., which contains 20-25 μM hemoglobin on a tetramer basis, 50 μM serum albumin, and 0.5 mM of the allosteric effector molecule, relative to the buffered red cell suspension without 50 μM serum albumin, and (b) maintains greater than eighty percent of its activity in terms of a calculated oxygen delivery index for the buffered red cell suspension containing 50 μM serum albumin relative to the buffered red cell suspension without 50 μM serum albumin; and permitting the allosteric effector molecule to penetrate into erythrocytes in the blood and bind to the hemoglobin therein.

Also included as allosteric effector compounds useful in the present invention are compounds disclosed in U.S. Pat. Nos. 5,677,330, 5,705,521 and 5,927,283 including a compound of the formula R₁—A—R₂ where R₁ and R₂ each are a substituted or unsubstituted aromatic or heteroaromatic compound, or substituted or unsubstituted alkyl or heteroalkyl ring compound, or a substituted or unsubstituted phthalimide compound, and where R₁ and R₂ may be the same or different, where A is a chemical bridge which includes two to four chemical moieties bonded together between R₁ and R₂, wherein said chemical moieties in A are selected from the group consisting of CO, O, S, SO₂, NH, NR₃ where R₃ is a C₁₋₆ alkyl group, NR₄ where R₄ includes two carbonyls as part of a phthalimide compound formed with R₁ or R₂, CH₂, CH, and C, with the caveat that, except in the case where A contains two identical CH and C moieties positioned adjacent one another to form an alkene or alkyne, the chemical moieties in A are each different from one another, and wherein at least one of R₁ or R₂ is substituted with a compound having the chemical formula:

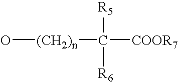

where n is zero to five, where R₅ and R₆ are selected from the group consisting of hydrogen, halogen, substituted or unsubstituted C₁₋₁₂ alkyl groups, carboxylic acid and ester, substituted or unsubstituted aromatic or heteroaromatic groups, and these moieties may be the same or different, or alkyl moieties of part of an aliphatic ring connecting R₅ and R₆, and where R₇ is a hydrogen, halogen, salt cation, metal, or substituted or unsubstituted C₁₋₆ alkyl group.

Also included as allosteric effector compounds useful in the present invention are compounds disclosed in U.S. Pat. No. 5,731,454 including

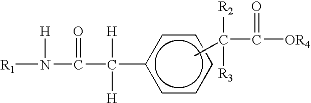

where R₁ is selected from the group consisting of optionally substituted phenyl, adamantyl, napthyl, and indanyl, R₂₋₃ are alkyl moieties of a C₃₋₆ alkyl ring connecting R₂ and R₃, and R₄ is a hydrogen, a monovalent salt cation, or a C₁₋₃ lower alkyl. Each of the above named patents, and all other patents and publications referred to herein, are incorporated by reference herein in their entirety.

In a preferred embodiment, the allosteric effector compound is 2-[4-[2-[(3,5-dimethylphenyl)amino]-2-oxoethyl]phenoxy]-2-methyl-propionic acid, which has the following structure:

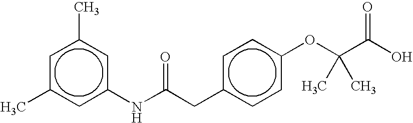

The sodium salt of 2-[4-[2-[(3,5-dimethylphenyl)amino]-2-oxoethyl]phenoxy]-2-methyl-propionic acid (C₂₀H₂₂NO₄Na; Molecular Weight=363.38) has the following structure:

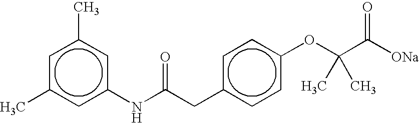

These compounds may be used in the composition in its acid form or in the form of a physiologically acceptable salt. The physiologically acceptable salt of 2-[4-[2-[(3,5-dimethylphenyl)amino]-2-oxoethyl]phenoxy]-2-methyl-propionic acid can be represented as having the following general structure where X⁺ represents the cation of the physiologically acceptable salt:

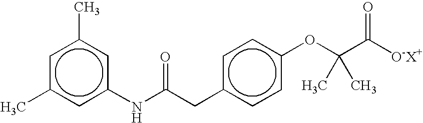

The salt may include compounds with inorganic or organic cationic counterions. For example, inorganic counterions may include, but are not limited to, sodium, potassium, calcium, magnesium, zinc, and combinations thereof Organic counterions may include, but are not limited to, lysine, hydroxy-lysine, histidine, arginine, ornithine, tromethamine, meglumine, and combinations thereof.

The allosteric modifying compound is preferably placed in solution prior to administration. The solution may be made using water, a saline solution, a dextrose solution, a lactated Ringer's solution, an aqueous solution of mannitol, or combinations thereof as the diluent. Other diluents may be used as long as they are suitable for parenteral administration to a patient. Preferably, the diluent does not reduce the chemical or physical (particle) stability of the allosteric modifying compound such that it fails the (USP) 25 <788>requirement.

Parenteral products must meet certain requirements for subvisual particulate matter. Failure to meet these requirements may result in the product being unacceptable for therapeutic treatment. The USP <788> provides standards for determining subvisual particulate matter. Two tests are provided, a light obscuration particle count test, and a microscopic particle count test. If the injection fails the light obscuration test, then it must pass the microscopic procedure. Alternatively, if a preparation can not be tested by light obscuration for technical reasons, e.g., high viscosity, microscopic testing can be used exclusively. For small volume injections of not more than 100 ml, the USP 25 <788> light obscuration limit for particles ≧10 microns is not more than 6000 per vial and for particles ≧25 microns the limit is not more than 600 per vial. For large volume injections, greater than 100 ml, the USP 25 <788> light obscuration limit for ≧10 micron particles is not more than 25 per ml and the limit for 25 micron particles is not more than 3 per ml. Thus, in some embodiments, the size of the container determines the total number of particles that may be present. For example, for a 100 mL container (defined by the USP as a small volume injectable), the requirement is 6 particles per milliliter of particles larger than ≧25 μm and not more than 60 particles per milliliter of particles ≧10 μm.

For small volume injections of not more than 100 ml, the microscopic limit for particles ≧10 microns is 3000 per vial. The USP 25 microscopic limit for particles ≧25 microns for small volume injections is 300 per vial. For larger volume injections, greater than 100 ml, the USP 24 microscopic limit for ≧10 micron particles is not more than 12 per ml and the limit for ≧25 micron particles is not more than 2 per ml.

In one embodiment where the composition will be used for treating conditions mediated through allosterically modifying hemoglobin, the composition preferably contains an amount of the allosteric modifying compound that is effective for allosterically modifying hemoglobin.

Preferably, the composition of the present invention comprises an amount ranging from about 15 millimoles/L to about 150 millimoles/L of the allosteric modifying compound. More preferably, the amount ranges from about 45 millimoles/L to about 90 millimoles/L of the allosteric modifying compound. In the most preferred embodiments, the composition of the present invention comprises about 58.7 mmol/L of the allosteric modifying compound. The amount of the allosteric modifying compound added can vary and depends on factors known to one skilled in the art. Factors may include the condition to be treated as well as the size and health of the patient.

It has been found that a formulation of the allosteric effector compound 2-[4-[2-[(3,5-dimethylphenyl)amino]-2-oxoethyl]phenoxy]-2-methyl-propionic acid at 20 mg/mL (58.7 mmol/L) at pH 7.5, not including a stabilizing compound, that was heat sterilized developed a precipitate of the allosteric modifier within one week after sterilization. While the allosteric modifying compound in this composition was stable to chemical degradation, this formation of particulate matter may result in the preparation failing the USP requirements. Interestingly, the formation of particulate matter takes place even though the concentration of the allosteric modifying compound is less than half of the solubility limit for the compound at the pH of the solution. The pKa for 2-[4-[2-[(3,5-dimethylphenyl)amino]-2-oxoethyl]phenoxy]-2-methyl-propionic acid is about 3.5. Accordingly, there is an appreciable solubility at a pH of 7. The solubility of the sodium salt of 2-[4-[2-[(3,5-dimethylphenyl)amino]-2-oxoethyl]phenoxy]-2-methyl-propionic acid at a pH of 7 is about 50 mg/ml. Surprisingly, 2-[4-[2-[(3,5-dimethylphenyl)amino]-2-oxoethyl]phenoxy]-2-methyl-propionic acid begins to precipitate out of solution, forming subvisual particulate where the concentration of 2-[4-[2-[(3,5-dimethylphenyl)amino]-2-oxoethyl]phenoxy]-2-methyl-propionic acid is only 20 mg/ml and the pH of the solution is about 7 at 25° C. Not being limited by this theory, these solubility studies suggest that this compound may be surface active, and undergoing a phase transition from a monomeric form to some sort of associated species, such as a small aggregate, oligomer or micelle, which solubilizes traces of the unionized acid. It is thought that the addition of a stabilizing agent maintains the integrity of this small aggregate-like species.

This unforeseen problem of formation of particulate matter at concentrations beneath the solubility of the compound is solved by the addition of a stabilizing agent. The effect of the stabilizing agent is to prevent or minimize the formation of sub-visual particulates. In some embodiments, the stabilizing agent may also act as a buffer to stabilize the pH of the solution. In other embodiments, the stabilizing agent and buffering agent are different. The stabilizing agent acts to prevent the formation of significant amounts of sub-visual particulate matter, particularly after heat sterilization. The result is a formulation that can be terminally sterilized, have a long shelf life, and meet the USP 25 <788> sub-visual particulate matter requirements. Without being bound by theory, it is believed that stabilization of pH is one factor contributing to stabilization of the formulation; however, the pH of the solution alone is insufficient to stabilize the solutions. Studies have shown that the pH of the allosteric effector compound should be greater than about 6.6 for optimum solubility. If necessary, the pH of the solution can be adjusted to a pH of at least about 6, preferably from about 6 to about 11. More preferably, the pH is adjusted to about 6.5 to about 9.0. More preferably, the pH is adjusted to about 7.5 to 8.5. The pH may be adjusted by the addition of any appropriate acid or base. Suitable acids may be amino acids, carboxylic acids, phosphoric acid, hydrochloric acid or other acids suitable for pharmaceutical preparations. Suitable bases include, sodium hydroxide or other bases suitable for pharmaceutical preparations.

The composition of the present invention includes a stabilizing agent. The stabilizing agent may minimize pH drift, but more importantly, the stabilizing agent acts to inhibit the formation of particulate matter in the composition. The stabilizing agent may be added to the composition as an additional component or, where the counter ion of a physiologically acceptable salt of the allosteric modifying compound being used has the capacity to act as a stabilizing agent, the counter ion itself may serve as the stabilizing agent. Without being bound by theory, one possible mechanism that allows the stabilizing agent to prevent particulate formation is that the stabilizing agent acts as a proton “sink” that lowers the probability of the formation of the less soluble neutral protonated allosteric modifier.

The selection of the stabilizing agent may depend, in part, on the final pH desired. The amount of the stabilizing agent will vary depending upon several factors known to those skilled in the art. Some of these factors include the composition of the stabilizing agent, the pKa(s) of the stabilizing agent, the concentration of the allosteric modifying compound, the amount of the solution to be stabilized, and the sterilization cycle used. The amounts and factors may vary from one stabilizing agent to the next. In any event, the amount of the stabilizing agent added to the composition should be an amount that is effective to reduce the formation of particulate matter in the composition. Further, the amount of stabilizing agent may preferably be an amount that maintains the pH of the composition within a desired range.

Suitable stabilizing agents include, but are not limited to, orthophosphoric acid, physiologically acceptable salts of orthophosphoric acid, citric acid, physiologically acceptable salts of citric acid, tromethamine, meglumine, amino acids, di-peptides, tri-peptides, glycine, glycyl-glycine, lysine, arginine, and other compounds containing an amine group, and combinations thereof.

In one embodiment, the stabilizing agent is orthophosphoric acid at a concentration of about 1-5 mM and the formulation has a pH of about 7.5, 8.0, or 8.5. In another embodiment, the stabilizing agent is tromethamine at a concentration of about 1-5 mM and the formulation has a pH of about 7.5, 8.0, or 8.5.

As a result of the investigation of the unexpected precipitate in formulations of 2-[4-[2-[(3,5-dimethylphenyl)amino]-2-oxoethyl]phenoxy]-2-methyl-propionic acid, it has been discovered that the concentration of the stabilizing agent and the solubility are surprisingly related in some cases. For example, an increase in solubility of approximately 12 mg/mL was found in 100-200 mM meglumine solutions at 23° C. and a pH of 7.5. The solubility of 2-[4-[2-[(3,5-dimethylphenyl)amino]-2-oxoethyl]phenoxy]-2-methyl-propionic acid increased steadily from approximately 44 mg/mL in water to 99.89 mg/mL in a 0.5 M tris solution at 23° C. and a pH of 7-8. Finally, the solubility of 2-[4-[2-[(3,5-dimethylphenyl)amino]-2-oxoethyl]phenoxy]-2-methyl-propionic acid increased significantly, from approximately 44 mg/mL in water to 155.07 mg/mL in a 0.5 M arginine solution at 23° C. and a pH of 7.2-7.5. Accordingly, stabilizing agents having an amine group are contemplated within the scope of this invention.

The composition of the present invention may be prepared by adding the allosteric modifying compound to an appropriate diluent and stabilizing agent. As discussed above, suitable diluents include, but are not limited to, water, a saline solution, a dextrose solution, lactated Ringer's solution, an aqueous solution of mannitol, and combinations thereof.

Where the stabilizing agent is not the counter ion of a salt of the allosteric modifying compound, the stabilizing agent is added to the solution as a separate component. The order in which the stabilizing agent, the allosteric modifying compound, and the pH adjuster is added is not critical. The stabilizing agent may be added to the liquid before or after the addition of the allosteric modifying compound.

Once the composition is prepared, it may be filled into a container. Alternatively, the preparation of the composition may occur in the container. Where the preparation is for intravenous administration, the composition may be prepared in the intravenous bag or bottle containing the intravenous solution.

Preferably, the composition should be sterile for administration. The preparation of the pharmaceutical composition may be made in a sterile environment. Any sterilization method that does not change the chemical composition of the allosteric modifying compound or induce particulate formation to the point where the pharmaceutical composition would fail the USP 24 <788>requirements may be used. Suitable methods may include, but are not limited to, sterile filling the composition into a sterile container, filling a container with the composition followed by heat sterilization, filter sterilization prior to filing the container; sterile filling the composition into a sterile container and heat sterilization.

The stabilized formulations of the present invention are stabilized for varying time periods. In one embodiment, the formulation is stabilized for at least about two weeks. In another embodiment, the formulation is stabilized for at least about 30 days. In a further embodiment, the formulation is stabilized for at least about six months. In yet a further embodiment, the formulation is stabilized for at least about one year. In yet a further embodiment, the formulation is stabilized for at least about two years.

The composition in accordance with the present invention has reduced particulate matter in solution and is suitable for parenteral routes of administration, including but not limited to, intravenous injection, continuous infusion, subcutaneous injection, intramuscular injection, and intraperitoneal injection.

The allosteric modifying compound is chemically and physically stable between a pH of about 6 and about 11. Preferably, the composition has a pH of at least about 6. More preferably, the composition has a pH ranging from about 6 to about 11. Most preferably, the composition has a pH ranging from about 6.5 to about 9.0.

As illustrated in Table I, the presence of a stabilizing agent significantly reduces the number of particles forming in the solution after terminal sterilization. All preparations were made at a concentration that was under half of their solubility limits.

TABLE I
Instrumental Particulate Matter observed Immediately After
Terminal Sterilization for Four Formulations of 59 millimoles/
L (20 mg/mL) 2-[4-[2-[(3,5-dimethylphenyl)amino]-2-
oxoethyl]phenoxy]-2-methyl-propionic acid in 0.225% NaCl
with the following stabilizing agents:

| Stabilizing Agent | ≧10μ (counts/mL) | ≧25μ (counts/mL) |
|---|---|---|
| USP Limit for Large | NMT 25 | NMT 3 |
| Volume Injectables | | |
| None | 140 | 6 |
| Phosphate | 2 | 0 |
| Glycyl-Glycine | 4 | 0 |
| Tromethamine | 13 | 0 |

The composition of the present invention is chemically stable and stable with respect to the formation of particulate matter for at least about thirty days. This makes the composition particularly useful at the administration site because no additional steps, such as filtering the composition, are necessary prior to administration to a patient. Stability data is provided in Table II. Time is time after sterilization.

| | ≧10μ (counts/mL) | | | ≧25μ (counts/mL) | | |
|---|---|---|---|---|---|---|
| USP Limit for Large | NMT 25 | | | NMT 3 | | |
| Volume Injectables | | | | | | |
| Stabilizing Agent\Time | 0 | 6 mo. | 12 mo. | 0 | 6 mo. | 12 mo. |
| Phosphate 1 mM | 3 | 20 | 4 | 0 | 0 | 0 |
| Glycyl-Glycine 1 nM | 4 | 4 | 4 | 0 | 0 | 0 |
| Tromethamine 1 mM | 12 | 10 | 9 | 0 | 0 | 0 |

It will be readily understood by those persons skilled in the art that the present invention is susceptible to broad utility and application. Many embodiments and adaptations of the present invention other than those herein described, as well as many variations, modifications and equivalent arrangement, will be apparent from or reasonably suggested by the present invention and the foregoing description without departing from the substance or scope of the present invention.

The foregoing disclosure is not intended to be construed to limit the present invention or otherwise exclude other embodiments, adaptations, variations, modifications or equivalent arrangements, the present invention being limited only by the appended claims and their equivalents.

What is claimed is:

1. A stabilized pharmaceutical composition comprising an allosteric modifier compound and a stabilizing compound.

2. The pharmaceutical composition of claim 1, wherein the allosteric effector compound is 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid.

3. The pharmaceutical composition of claim 1, wherein the composition has not more than 3 particles per milliliter of particles ≧25 μm and not more than 25 particles per milliliter of particles ≧10 μm.

4. The pharmaceutical composition of claim 1, wherein the composition has not more than 600 particles per container of particles ≧25 μm and not more than 6000 particles per containerof particles ≧10 μm.

5. The pharmaceutical composition of claim 1, wherein the composition has not more than 2 particles per milliliter of particles ≧25 μm and not more than 12 particles per milliliter of particles ≧10 μm.

6. The pharmaceutical composition of claim 1, wherein the composition has not more than 300 particles per container of particles ≧25 μm and not more than 25 particles per milliliter of particles ≧10 μm.

7. The composition of claim 2, comprising an amount of 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid ranging from about 15 millimoles/L to about 120 millimoles/L.

8. The composition of claim 2, comprising an amount of 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid ranging from about 45 millimoles/L to about 90 millimoles/L.

9. The composition of claim 1 wherein the stabilizing agent is selected from the group consisting of orthophosphoric acid, physiologically acceptable salts of orthophosphoric acid, citric acid, physiologically acceptable salts of citric acid, tromethamine, meglumine, amino acids, di-peptides, tri-peptides, glycine, lysine, arginine, glycyl-glycine, and combinations thereof.

10. The composition of claim 2, wherein 2-[4-[2-[(3,5-dimethylphenyl)amino]-2-oxoethyl]phenoxy]-2-methyl-propionic acid is present as a physiologically acceptable salt selected from the group consisting of sodium, potassium, calcium, magnesium, zinc, and combinations thereof.

11. The composition of claim 2 wherein 2-[4-[2-[(3,5-dimethylphenyl)amino]-2-oxoethyl]phenoxy]-2-methyl-propionic acid is present as a physiologically acceptable salt of a compound containing an amine group.

12. The composition of claim 11 wherein the compound containing an amine group is selected from the group consisting of lysine, hydroxy-lysine, histidine, arginine, ornithine, tromethamine, meglumine, and combinations thereof.

13. The composition of claim 2, comprising an amount of 2-[4-[2-[(3,5-dimethylphenyl)amino]-2-oxoethyl]phenoxy]-2-methyl-propionic acid effective for the treatment of conditions mediated through allosterically modifying hemoglobin to shift oxygen equilibrium in favor of free oxygen.

14. The composition of claim 1, comprising a physiologically acceptable salt of 2-[4-[2-[(3,5-dimethylphenyl)amino]-2-oxoethyl]phenoxy]-2-methyl-propionic acid having a counter ion, and wherein the counter ion acts as the stabilizing agent.

15. The composition of claim 1 wherein the composition is sterile.

16. The composition of claim 1, further comprising a diluent, wherein said diluent is selected from the group consisting of water, a saline solution, a dextrose solution, lactated Ringer's solution, an aqueous solution of mannitol, glucose polymers, modified glucose polymers, and combinations thereof.

17. A process of making a pharmaceutical composition of 2-[4-[2-[(3,5-dimethylphenyl)amino]-2-oxoethyl]phenoxy]-2-methyl-propionic acid, comprising the steps of combining 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid and a stabilizing agent.

18. The process of claim 17, wherein said 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid is provided in a diluent.

19. The process of claim 18, wherein the diluent has a pH above about 6.6.

20. The process of claim 17, wherein the stabilizing agent is added in amount sufficient to minimize the formation of particulates in the pharmaceutical composition.

21. The process of claim 17, wherein the stabilizing agent maintains the composition having not more than 3 particles per milliliter of particles ≧25 μm and not more than 25 particles per milliliter of particles ≧10 μm.

22. The process of claim 17, wherein the stabilizing agent maintains the composition having not more than 6 particles per milliliter of particles ≧25 μm and not more than 60 particles per milliliter of particles ≧10 μm.

23. The process of claim 17 wherein the stabilizing agent maintains the pH of the composition from about 6.5 to about 11.

24. The process of claim 17 wherein the stabilizing agent maintains the pH of the composition from about 6.5 to about 9.0.

25. The process of claim 17 wherein 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid is added in an amount ranging from about 15 millimoles/L to about 120 millimoles/L.

26. The process of claim 17 wherein 2-[4-[2-[(3,5-dimethylphenyl)amino]-2-oxoethyl]phenoxy]-2-methyl-propionic acid or at least one physiologically acceptable salt of 2-[4-[2-[(3,5-dimethylphenyl)amino]-2-oxoethyl]phenoxy]-2-methyl-propionic acid is added in an amount ranging from about 45 millimoles/L to about 90 millimoles/L.

27. The process of claim 17 wherein the buffer is selected from the group consisting of orthophosphoric acid, physiologically acceptable salts of orthophosphoric acid, citric acid, physiologically acceptable salts of citric acid, tromethamine, meglumine, amino acids, di-peptides, tri-peptides, glycine, lysine, arginine, glycyl-glycine, and combinations thereof.

28. The process of claim 17 wherein 2-[4-[2-[(3,5-dimethylphenyl)amino]-2-oxoethyl]phenoxy]-2-methyl-propionic acid is present as a physiologically acceptable salt selected from the group consisting of sodium, potassium, calcium, magnesium, zinc, and combination thereof.

29. The process of claim 17 wherein 2-[4-[2-[(3,5-dimethylphenyl)amino]-2-oxoethyl]phenoxy]-2-methyl-propionic acid is present as a physiologically acceptable salt selected from the group consisting of lysine, hydroxy-lysine, histidine, arginine, ornithine, protonated tromethamine, meglumine, and combinations thereof.

30. The process of claim 17 further comprising the step of sterilizing the composition.

31. The process of claim 30 wherein the sterilizing step is performed by heat sterilization.

32. The process of claim 17 further comprising sterile filling the composition into a sterile container.

33. The process of claim 18 wherein the diluent is selected from the group consisting of water, saline solution, dextrose solution, lactated Ringer's solution, an aqueous solution of mannitol, glucose polymers, modified glucose polymers, and combinations thereof.

34. A method of allosterically modifying hemoglobin, comprising administering to a patient in need thereof a stabilized pharmaceutical composition of claim 1.

35. A method for measuring a blood oxygen level-dependent magnetic resonance imaging signal, comprising
a) administering a stabilized pharmaceutical composition of claim 1; and
b) performing a blood oxygen level-dependent magnetic resonance imaging scan, whereby said blood oxygen level-dependent magnetic resonance imaging signal is measured.

36. A method of increasing the sensitivity of cells to the cytotoxic effects of ionizing radiation comprising:
a) contacting the cells with stabilized pharmaceutical composition of claim 1to oxygenate the cells; and
b) administering an effective cytotoxic dose of ionizing radiation to the cells.
